(19) United States
(12) Patent Application Publication
(10) Pub. No.: US 2023/0321361 A1
(43) Pub. Date: Oct. 12, 2023

(54) Medical syringe with passive needle protection

(71) Applicant: TRENTA2 S.R.L., Bozen (IT)

(72) Inventor: Dietrich Gallmetzer, Terlan (IT)

(73) Assignee: TRENTA2 S.R.L., Bozen (IT)

(21) Appl. No.: 18/025,128

(22) PCT Filed: Sep. 8, 2020
(86) PCT No.: PCT/EP2020/075087

§ 371 Date: Mar. 7, 2023

(51) Int. Cl. A61M 5/32

(52) U.S. Cl. CPC A61M 5/3234; A61M 2005/31516

(57) ABSTRACT

A syringe with passive needle protection for administering a medicament dose to a patient, comprising a hollow body forming the syringe housing and containing the medicament active substance, at the distal end of which a hollow needle provided for injection of the active substance is mounted in a bearing sleeve, and in which a displaceable plunger provided at the end with a receiving hole for the hollow needle is arranged, for the most complete possible delivery of medicament while avoiding undelivered medicament residues. For this purpose, the piston is designed in several parts; in addition to a needle holder having a retaining clip for the hollow needle, it comprises a piston jacket which is shaped such that, when the needle holder is inserted, it leaves free inflow channels into the enclosed end of the hollow needle for the active substance on both sides of the retaining clip.

### CROSS-REFERENCE TO RELATED APPLICATIONS

This application is a national phase of the international application PCT/EP2020/075087, filed Sep. 8, 2020 and titled “MEDIZINISCHE SPRITZE MIT PASSIVEM NADELSCHUTZ” (“MEDICAL SYRINGE WITH PASSIVE NEEDLE GUARD/PROTECTION”), which is hereby incorporated by reference in its entirety.

### FIELD OF INVENTION

The invention relates to a medical syringe with passive needle protection for administering a dose of medication to a patient, with a hollow body forming the syringe housing and containing the medical active substance, at the distal end of which a hollow needle intended for injecting the active substance is mounted in a bearing sleeve, and in which a displaceable plunger provided at the end with a receiving hole for the hollow needle is arranged.

### BACKGROUND

To avoid or reduce the risk of contamination or injury after the use of medicinal syringes and in particular to avoid multiple use of syringe needles by different users, syringes with so-called retraction or retraction systems for the syringe needle are increasingly used. In such syringes, in particular medical syringes, also known as “tips with passive needle protection”, the syringe needle is retracted into the syringe body after dispensing the active substance held in the syringe and is completely enclosed by the syringe body. Access to the syringe and thus a risk of injury, or even the risk of multiple use of the same needle, can thus be largely excluded.

Such syringes with passive needle protection are known, for example, from EP 1 284 769 B1, EP 0 720 491 B1, EP 0 680 347 B1 or EP 1 764 127 B1.

The invention is now based on the task of further designing a syringe with passive needle protection of the above-mentioned type in such a way that it is possible to deliver the medicament contained in the syringe body as completely and as far as possible, while largely avoiding any residual medication remaining in the syringe body after delivery.

This task is solved according to the invention in that the plunger is made in several parts and, in addition to a needle holder having a retaining clip for the hollow needle, comprises a plunger jacket which is shaped in such a way that, when the needle holder is inserted, it leaves free inflow channels on both sides of the retaining clip into the enclosed end of the hollow needle for the active substance.

The invention is based on the consideration that for the desired reduction or, if possible, complete avoidance of drug residues remaining in the syringe, the dead volumes in the drug chamber and the flow channels for the drug should be kept as small as possible. Particularly in the case of syringes with a retraction system, however, the hollow needle must usually be coupled to the syringe plunger towards the end of the dispensing process so that it can be drawn into the syringe body via the plunger during retraction. This usually requires a mechanical enclosure of the hollow needle, which on the other hand causes the formation of inherently undesirable dead volumes. To counteract this, the syringe plunger is now specifically designed to offer suitable flow paths for the active substance even after contact with the hollow needle, through which it can still be supplied to the needle even in this state.

Advantageously, and for a particularly cost-effective design, the retaining bracket of the needle holder carries a needle bearing which forms the receiving hole and is provided on the inside with a circumferential latching lip which, when the hollow needle is pushed into the needle bearing, engages in a retaining groove arranged on its outer circumference and fixes it in the longitudinal direction.

In order to ensure that during the retraction movement of the plunger the needle is carried along by the plunger towards the interior of the syringe body and thus retracted into the syringe housing, the dimensions of the retaining groove and its associated locking lip in the needle bearing are advantageously selected in such a way, that, taking into account the deformability of the material on the outer circumference of the hollow needle and the material of the needle bearing surrounding it, the holding or breakaway force of the needle thus engaged in the needle bearing is greater in the longitudinal direction than the corresponding holding or breakaway force of a holding groove of the hollow needle in the bearing sleeve.

In order to ensure even further improved reliability and operating safety of the syringe during use, the syringe is in a very particularly preferred embodiment provided with a retraction-triggering spring which, when the needle is retracted into the interior of the syringe body, additionally also displaces the trigger plate provided for actuating the syringe in a direction away from the distal end of the syringe. As a result, the intended retraction or retraction of the syringe needle into the syringe body can be perceived by the user not only visually, but also in other usable ways, in particular by means of haptic perceptibility. In particular, the syringe, coupled to the retraction process of the needle tip, generates a corresponding displacement of its associated trigger plate, which can be felt by the user, for example, via the thumb. In this way, without the user having to actively observe the syringe, the information can be transmitted to the user that the needle has been properly retracted and, above all, that the active substance has been completely administered and the delivery of the active substance has thus ended.

### BRIEF DESCRIPTION OF THE DRAWINGS

An embodiment of the invention is explained in more detail with reference to a drawing. Therein show:

FIG. 1 a medical syringe with passive needle protection,

FIG. 2 a cartridge unit of the syringe according to FIG. 1 in side view,

FIG. 3 the cartridge unit according to FIG. 2 in partial section,

FIG. 4 an exploded view of the cartridge unit according to FIG. 2,

FIG. 5 an actuation unit of the syringe according to FIG. 1 in side view,

FIG. 6 the actuation unit according to FIG. 5 in partial section,

FIG. 7 an exploded view of the actuation unit according to FIG. 5,

FIG. 8 a sequence of partial sections of the syringe according to FIG. 1,

FIG. 9 a side view of a needle inserted into a needle holder for use in the syringe according to FIG. 1,

FIG. 10 a perspective view of the needle as shown in FIG. 9,

FIG. 11 a plunger of the syringe according to FIG. 1,

FIG. 12 an actuating plunger of the syringe according to FIG. 1,

FIG. 13 the actuating plunger according to FIG. 12 in longitudinal section, and

FIG. 14,15 each an alternative embodiment of a medical syringe with needle protection and double chamber system in longitudinal section.

Identical parts are marked with the same reference signs in all figures.

### DETAILED DESCRIPTION OF THE DRAWINGS

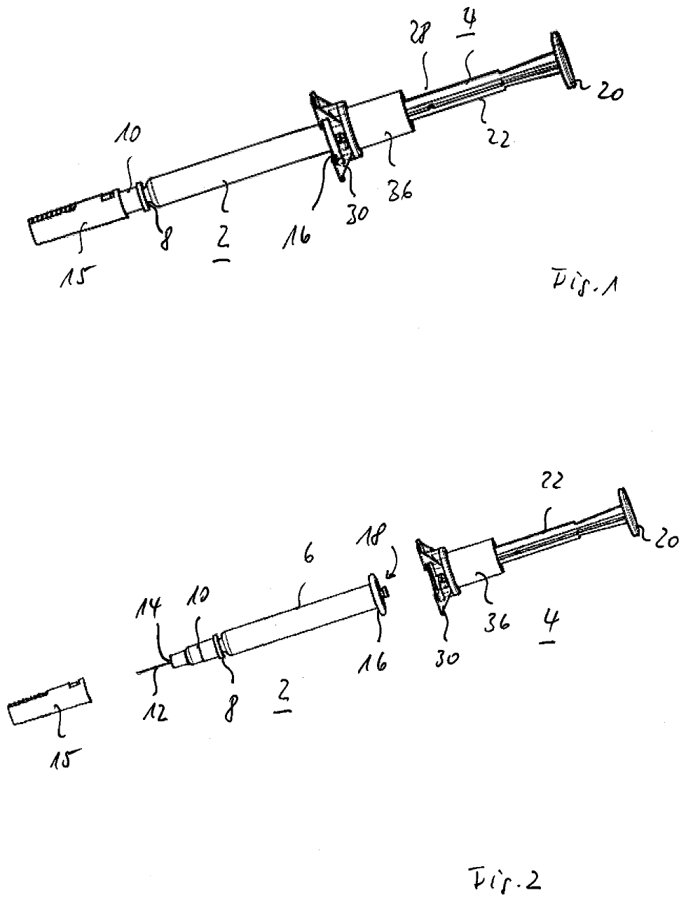

The medical syringe 1 with passive needle protection according to FIG. 1 essentially comprises two subassemblies, namely a cartridge unit 2 on the one hand and a drive or actuation unit 4 on the other. In the embodiment example, the medical syringe 1 has a two-component design, the two subassemblies mentioned forming separate components that can be connected to one another in the manner described below. Alternatively, the syringe could also have a single-component design, in which case the two assemblies mentioned are connected to each other from the outset and the distinction between the two assemblies is merely functional.

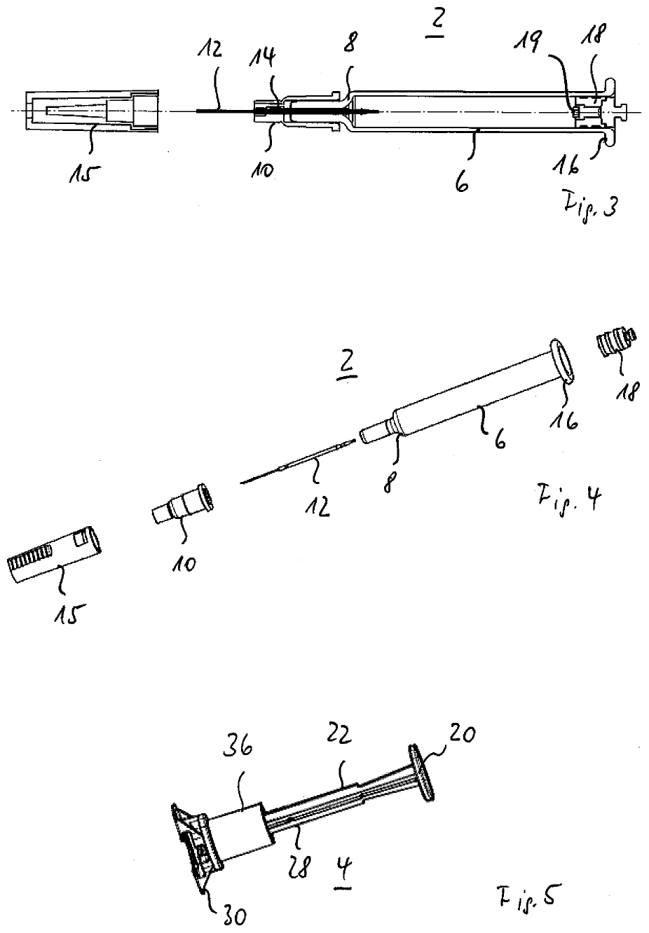

The first assembly, i.e. the cartridge or cartridge unit 2, shown in FIG. 2 in a state before administration of the medical active substance, in FIG. 3 in a partial section and in FIG. 4 in an exploded view, forms the actual syringe and comprises a hollow body 6 which is cylindrical or tubular in accordance with a conventional design and forms a syringe housing and into which the medical active substance is filled. A needle holder 10 is attached to the front or distal end 8 of the hollow body 6, in which the hollow needle 12 intended for injection of the active substance is mounted in a bearing sleeve 14. The needle holder 10 could be made in one piece with the hollow body 6 forming the syringe housing. In the embodiment example, however, the needle holder 10 is designed as a separate component in an embodiment considered to be independently inventive. The needle holder 10 is attached or attachable to the hollow body 6 forming the syringe housing or to the syringe cone, but could also be screwed on by means of a thread, for example a Luer thread.

The needle 12 is surrounded by a removable protective cap 15 to prevent injury or the like, which is removed before the syringe 1 is used. The rear or proximal end 16 of the hollow body 6 forming the syringe housing, on the other hand, is closed by a plunger 18 whose outer dimensions are precisely adapted to the inner contour of the hollow body 6 and which can be moved within the hollow body 6. In the state shown in FIG. 2, i.e. before the medicinal substance is dispensed, it is thus enclosed in the interior of the hollow body 6, which is closed at the end by the piston 18. On its end surface facing the interior of the hollow body 6, the piston 18 has a central receiving hole 19 for the needle 12.

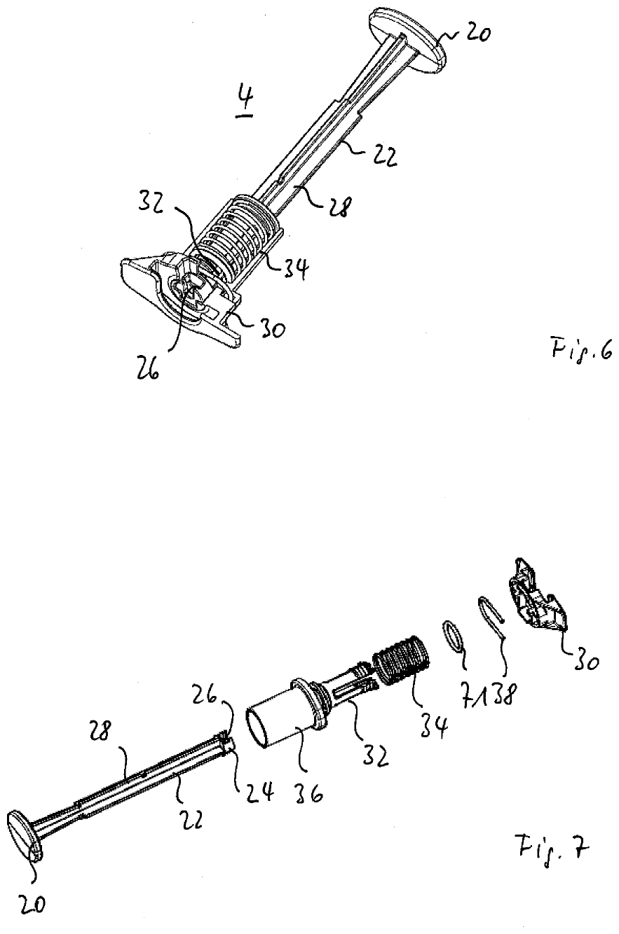

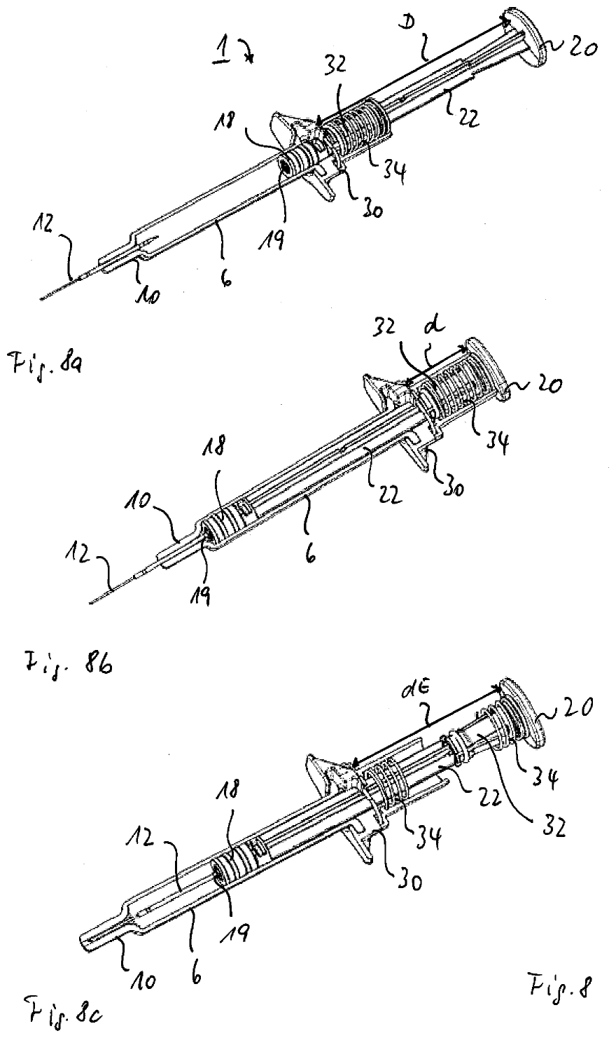

The second assembly of the medical syringe 1, i.e. the drive or actuation unit 4, shown in FIG. 5 in lateral view, in FIG. 6 in partial section and in FIG. 7 in exploded view, comprises an actuation plunger 22 provided at the end with a plunger plate 20 and designed as an elongated element or shaft-like element. At its end 24 opposite the plunger plate 20, the actuation plunger 22 has a coupling element 26 provided for connection to the piston 18. With its shaft 28 extending between the pusher plate 20 and the coupling element 26, the actuating plunger 22 is guided through a coupling plate 30 and is mounted in the latter so as to be displaceable in its longitudinal direction. On the side of the coupling plate 30 facing away from the connection with the cartridge unit 2, a locking element 32 is arranged surrounding the shaft 28 in a partial section, which in turn is surrounded by a compressed spring 34 which is under pretension in the state shown in FIG. 6 before the connection with the cartridge unit 2. These components are in turn positioned within a protective housing 36 designed in the manner of a tubular socket and attached to the dome plate 30. A safety spring 38 is also provided in the coupling plate 30 in the connection area to the protective housing 36.

The medical syringe 1 is provided with a passive needle protection in the form of a retraction system by the components and parts mentioned. The purpose of this is that after use of the syringe 1, i.e. after dispensing the active substance held in the hollow body 6 forming the syringe housing via the needle 12, the latter is drawn into the syringe housing in such a way that it is completely enclosed by the syringe housing. This is intended to keep unintentional contact with the used needle 12, for example by auxiliary or nursing personnel, and thus the risk of injury and contamination particularly low or, if possible, completely excluded.

For this purpose, the following procedure is basically intended for the use and handling of the components mentioned:

Due to the two-component design of the overall system provided in the embodiment example, the cartridge unit 2 and the actuation unit 4 are first suitably connected to each other. For this purpose, on the one hand the coupling element 26 is connected to the piston 18 and on the other hand the coupling plate 30 is connected to the proximal end 16 of the hollow body 6. In the case of an a priori single-component design of the overall system, in which, for example, the actuating plunger 22 could be connected directly to the piston 18 and the coupling plate 30 could be moulded directly onto the proximal end 16 of the hollow body 6, this connection step can naturally be omitted.

After the syringe 1 has been made ready for use in this way, the needle 12 is suitably positioned on the patient for the administration of the medical active substance so that it pierces the patient's skin at a suitable point. The retaining circle of the needle 12 in the bearing sleeve 14 is thereby predetermined, in particular by suitable dimensioning of the components and/or the choice of material pairing, in such a way that the needle 12 remains securely in its position in the bearing sleeve 14 when the operator pierces the needle 12 through the patient's skin by handling it on the hollow body 6.

The actuating plunger 22 is then pressed by the operator so that the piston 18 moves inside the hollow body 6 towards its distal end 8, thereby feeding the medicinal agent to the needle 12 and dispensing it via the latter. Shortly before complete dispensing of the active ingredient, i.e. shortly before complete emptying of the interior of the hollow body 6, the piston 18 reaches the end of the needle 12 projecting into the interior in the vicinity of the distal end 8 of the hollow body 6, so that the needle 12 enters the receiving hole 19 provided for this purpose during further movement. After complete dispensing of the active substance, the piston 18 then reaches its end position directly at the distal end 8 of the hollow body 6 and thereby encloses the part of the hollow needle 12 projecting into the receiving hole 19. This insertion of the corresponding part of the hollow needle 12 into the receiving hole 19 and the connection of the needle 12 with the piston 18 thereby achieved is also referred to herein as “connecting”.

As soon as this point is reached and the piston 18 has arrived at its intended end position, the locking element 32—in the embodiment example via a catch on the actuating plunger 22—is to deform suitably and thereby release the fixing of the spring 34. In the embodiment example, the dimensioning of the corresponding components is thereby selected in such a way that the “connecting” occurs slightly or briefly before the release of the spring 34; this is considered to be independently inventive. In response to this, i.e. to the release of the spring 34, the pre-tensioned spring 34 relaxes and thereby moves the pusher plate 20 of the actuating plunger 22 away from the coupling plate 30 and thus away from the syringe body formed by the hollow body 6. This also entrains the plunger 18, which is connected to the shaft 28 of the actuating plunger 22 via the coupling element 26, and pulls it within the hollow body 6 away from the distal end 8 towards the proximal end 16. In turn, it takes the enclosed needle 12 with it and pulls it into the hollow body 6 so that it is completely positioned inside the hollow body 6 in the final state.

A significant and considered inventive aspect in this respect is that the spring 34 provided for carrying out the retraction movement of the needle 12 into the hollow body 6 is arranged above the coupling plate 30 in the region of the shaft 28 of the actuating plunger 22 and acts on the actuating plunger 22 via the plunger plate 20 thereof. In contrast to conventional retraction systems, in which the retraction spring is arranged in the region of the tip or the distal end of the syringe within the syringe body and acts directly on the needle or its bearing body, the design now provided in accordance with the invention means that the needle 12 is moved together with the actuating plunger 22 and, in particular, its pusher plate 20 during retraction, so that the user or operator can detect the position and, in particular, the change in position of the needle 12 on the basis of the position of the pusher plate 20 relative to the coupling plate 30. This detection is possible visually, but above all also haptically.

In other words, the retraction movement, similar to an actuating button of a biro, produces a movement of the pusher plate 20 that can be felt by the user, for example with the thumb. Thus, the user can conclude from the movement of the pusher plate 20 that can be felt with the thumb, on the one hand, that the needle 12 has now been retracted and thus there is no longer any risk of injury or contamination in the area of the distal end 8 of the syringe 1. On the other hand, he can also conclude from this that the active substance has now been completely administered without having to monitor this visually, for example. The syringe 1 thus enables significantly simplified handling when administering the active substance.

For better clarification, this sequence is shown in the sequence of partial sections in FIG. 8. FIG. 8a shows the syringe 1 before the administration of the active substance contained in the interior of the hollow body 6. The plunger 18 guided in the interior of the hollow body 6 is here located at its stop on the coupling plate 30 arranged at the proximal end 16 of the hollow body 6. The actuating plunger 22 is accordingly fully extended, and the plunger plate 20 is located at the maximum distance D from the coupling plate 30. The spring 34 is held preloaded via the locking element 32 and positioned in the section of the fastening plunger 22 directly adjacent to the coupling plate 30. The hollow needle 12 held in the needle holder 10 is extended ready for use in this state.

Starting from this state, the active substance is dispensed—in particular when the needle 12 is inserted into the patient's skin—by moving the plunger 18 from its starting position shown in FIG. 8a to the end position shown in FIG. 8b immediately adjacent to the distal end 8 of the hollow body 6. For this purpose, the actuating plunger 22 is pushed into the hollow body 6 up to the end position in which the pusher plate 20 assumes the minimum distance d from the coupling plate 30. In this momentary state, the spring 34 is still preloaded, but due to the predetermined shape of the actuating plunger 22, the locking element 32 is released when this position is reached. The hollow needle 12 held in the needle holder 10 is still extended in this state, but has already entered the receiving opening 19 of the piston 18 and is enclosed by the piston 18 with its end projecting into the interior of the hollow body 6 and is mechanically connected to it.

When the piston 18 or the actuating plunger 22 reaches the end position shown in FIG. 8c, the locking element 32 is triggered so that it releases the pre-tensioned spring 34. In response, the coil spring 34 expands in its longitudinal direction and, being supported at the rear by the clutch plate 30, exerts a corresponding restoring force on the pusher plate 20, displacing it in the direction away from the clutch plate 30, to the end position shown in FIG. 8c. In this end position, the spring 34 is released and the pusher plate 20 is in a position with the end distance dE from the coupling plate 30. Accordingly, the actuating plunger 22 has moved along in the process, so that in this position the piston 18 is in a middle position inside the hollow body 6. The hollow needle 12 has been carried along by the piston 18 and is now completely inside the hollow body 6.

As can be seen from the illustrations in FIG. 8, the final distance dE is smaller than the original maximum distance D, but larger than the intermediate minimum distance d. This means that the pusher plate 20 automatically “extends” after the active substance has been completely dispensed and moves upwards, away from the pusher plate 20. This gives the user a haptic feedback signal, which can be felt with the thumb, for example, that the active ingredient has been completely dispensed and the needle is now secured in the interior of the hollow body.

In order to achieve the mode of operation explained above in a particularly reliable and advantageous manner in several respects, the components are specifically designed in various details, whereby the embodiments described below are each considered to be both independently inventive and inventive in any combination with one another.

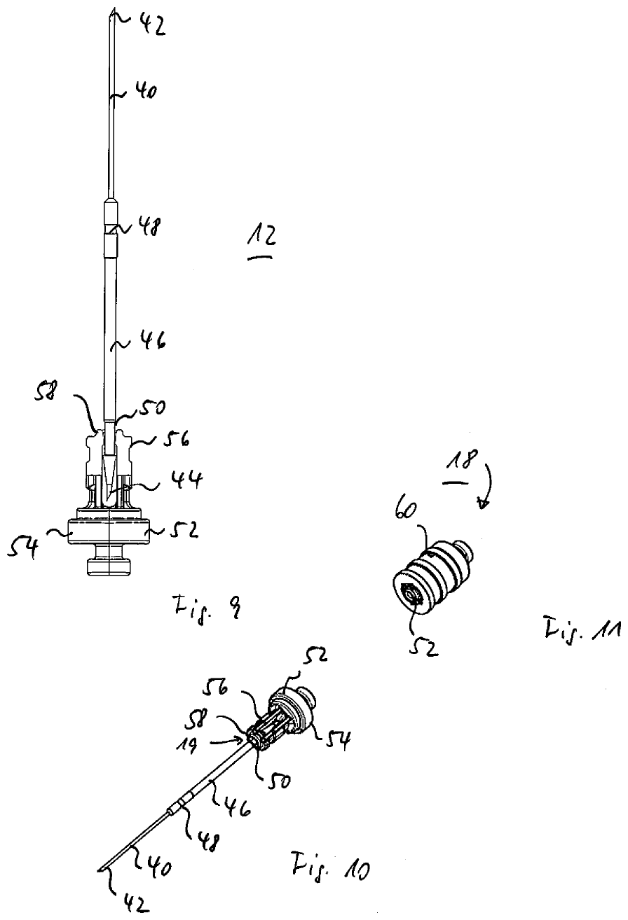

For example, the hollow needle 12 is independently inventive according to the following description. As can be seen from the enlarged representation in FIG. 9 (side view) and FIG. 10 (perspective view), the hollow needle 12 comprises, in an independently inventive design, a needle tube 40 made of metal, which forms a needle point 42, 44 at each of its two ends. The material for the needle tube 40 is preferably selected with regard to common requirements for medical applications, whereby a stainless material which can also be used for standard needles is particularly preferred. In its middle length range, the needle tube 40 is sheathed and surrounded by a plastic sheath 46. The material for the plastic sheath 46 is preferably a polyamide (PA12), most preferably the one commercially available under the designation Vestamid Care ML 17. In a very particularly preferred embodiment, which is also considered to be independently inventive, the plastic sheath 46 is sprayed onto the needle tube 40 after the latter has been subjected to a plasma pretreatment in the manner of a surface activation. In this way, a particularly good adhesion of the plastic forming the plastic sheath 46 to the needle 40 can be achieved. Two retaining grooves 48, 50 are formed in the plastic sheath 46 to enable the above-described mode of operation.

The first holding groove 48 is provided for temporarily fixing the needle 12 in the bearing sleeve 14 of the needle holder 10. For this purpose, an associated circumferential locking lip is provided inside the bearing sleeve 14, which engages in the holding groove 48 when the needle 12 is mounted and properly inserted into the bearing sleeve 14 and fixes it in the longitudinal direction. In accordance with an embodiment which is fundamentally considered to be independently inventive, the dimensions of the retaining groove 48 and the latching lip are thereby advantageously selected in such a way that, taking into account the deformability of the material of the plastic sheath 46 and/or any adhesive force due to the material pairing of the material of the plastic sheath 46 and the material of the bearing sleeve 14 surrounding it, the retaining or breakaway force of the needle 12 thus latched in the longitudinal direction is, on the one hand, sufficiently great, so that the needle 12 can be inserted into the patient's skin in accordance with the procedure described above, but on the other hand is also sufficiently small so that the described retraction movement of the needle 12 towards the interior of the hollow body 6 can be carried out. If necessary, the profile of the retaining groove 48 can also be designed accordingly asymmetrically, with a comparatively steep flank angle on its side facing the tip 44 facing the interior and a comparatively flat flank angle on its side facing the exposed tip 42.

The second retaining groove 50, on the other hand, is provided for a corresponding catch in the piston 18. In FIGS. 9, 10 the needle 12 is shown in the inserted state in the piston 18. The piston 18 is made of several parts and includes the needle holder 52 also shown in FIGS. 9, 10, which is considered to be independently inventive. The needle holder 52 is designed as a plastic part and, in the embodiment example, is made of polypropylene available under the designation “Bormed™” (HD810MO, ISO 10993 Information (Biocompatibility)) with regard to the required holding forces and the mechanical loads expected during intended use, but also with regard to approval-related requirements.

As can be seen in FIG. 9, and particularly well in the perspective view in FIG. 10, the needle holder 52 comprises a retaining bracket 56 moulded onto a base body 54, which carries the actual needle bearing 58 forming the receiving hole 19. The needle bearing 58, analogous to the bearing sleeve 14 described above, is provided on the inside with an associated circumferential latching lip which, when the needle 12 is pushed into the needle bearing 58, engages in the second retaining groove 50 and fixes it in the longitudinal direction. In accordance with an embodiment which is also regarded in principle as independently inventive, the dimensions of the retaining groove 50 and of the detent groove in the needle bearing 58 associated therewith are advantageously selected in such a way that, taking into account the deformability of the needle 12, the deformability of the needle bearing 58 is not impaired, that, taking into account the deformability of the material of the plastic jacket 46 and/or a possible adhesive force due to the material pairing of the material of the plastic jacket 46 and the material of the needle bearing 58 surrounding it, the holding or breakaway force of the needle 12 thus engaged in the longitudinal direction is greater than the corresponding holding or breakaway force of the holding groove 48 in the bearing sleeve 14, so that during the retraction movement of the piston 18 the needle 12 is carried along by the latter towards the interior of the hollow body 6.

The design of the retaining clip 56 also creates a free space inside the plunger 18 which, in the final phase of dispensing the active substance, when the needle tip 44 has already penetrated the receiving hole 19 and is therefore no longer readily accessible to the active substance, allows the active substance to flow into the needle tube 40 via the needle tip 44 in the manner of a bypass. The inflow can take place on both sides of the retaining clip 56 into the free space.

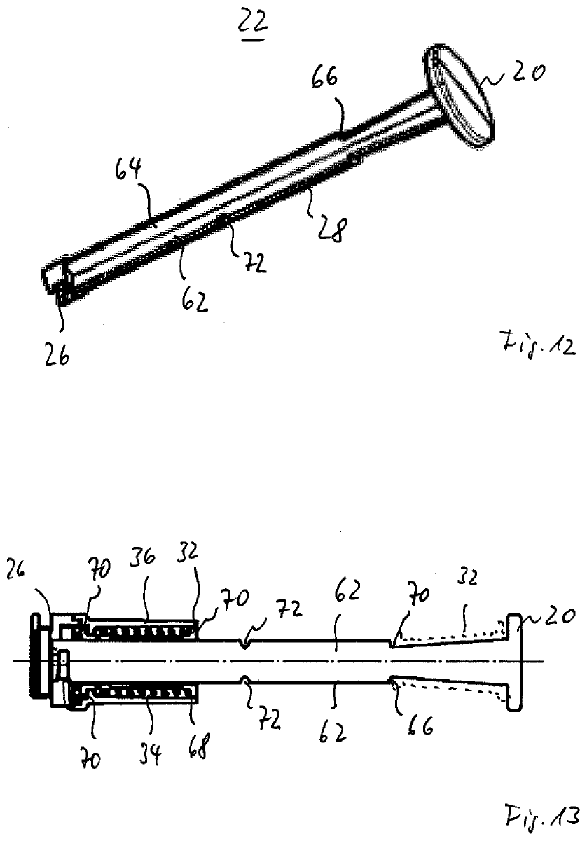

As already explained, in a further embodiment, which is also considered to be independently inventive, the piston 18 is made of several parts. As can be seen in the enlarged view according to FIG. 11, in addition to the already described needle holder 52 for the needle 12, a piston jacket 60 surrounding the needle 12 is provided. The piston skirt 60 is made of conventional rubber, particularly preferably of the material available from Kreiburg under the designation TM4RST (MC/RS Series), taking into account approval-related requirements. The piston jacket 60 is shaped in such a way that, when the needle holder 52 is inserted, it leaves free inflow channels for the active substance on both sides of the retaining clip 56, so that the bypass for the desired zero volume output is formed in the sense of avoiding or minimizing the dead volume.

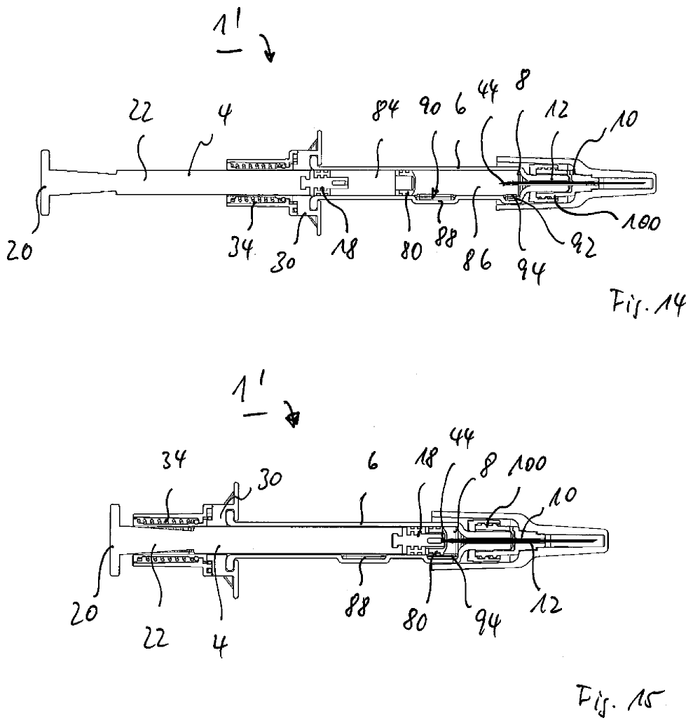

In order to enable the intended release of the locking element 32 for releasing the pre-tensioned spring 34, a specific design of the shaft 28 of the actuating plunger 22, which is also considered to be independently inventive, is provided. For clarification, this is shown enlarged in FIG. 12. As can be seen from this illustration, the shaft 28 is essentially formed by two crossed shaft ribs 62, 64 forming a cross in cross-section. As can be seen from the illustration in longitudinal section according to FIG. 13, the upper edges of the shaft ribs 62, 64, viewed in the longitudinal direction of the actuating plunger 22, run essentially in a straight line and over large parts of the overall length of the actuating plunger 22 essentially parallel to its longitudinal direction. However, at a position relatively close to the pusher plate 20, the shaft ribs 62, 64 have a recess 66 so that an edge is formed here.

In the ready-to-use state of the syringe 1, the locking element 32, which forms a number of pretensioned legs 68, is pushed onto the shaft 28 in the end region, as shown on the left in FIG. 13. The shaft ribs 62, 64 support the pre-tensioned legs 68 so that they remain straight. At the end, a support bead 70 is formed on the legs 68, on which the pre-tensioned spring 34 provided on the outside of the locking element 32 is supported. The support maintains the pre-tension of the spring 34.

If, as shown in FIG. 8b, the syringe 1 reaches the end position of the plunger 18 in the hollow body 6 when the active substance is administered, the locking element 32 sweeps over the entire length of the recess 66 of the shaft 28, so that the inward support of the pretensioned legs 68 no longer exists. As a result, the legs 68, driven by their pre-tension, give way inwards, as indicated in dashed form in FIG. 13 on the right. This also causes the support beads 70 to move inwards, so that the spring 34 is no longer supported and can relax. This initiates the desired retraction.

Furthermore, a notch 72 is also provided in the shaft ribs 62, 64, into which the securing spring 38 arranged in the dome plate 30 can engage when positioned appropriately. This serves to fix the assumed position after retraction of the needle 12 into the interior of the hollow body 6, so that the needle 12 remains secured there.

An alternative embodiment of a medical syringe 1′ with passive needle protection of the type mentioned, which is also considered to be independently inventive, is shown in longitudinal section in FIGS. 14, 15. FIG. 14 shows the medical syringe 1′ before, FIG. 15 after dispensing of the active substance shortly before triggering of the retraction mechanism and retraction of the needle 12. In this alternative embodiment, the medical syringe 1′ is equipped with a double chamber or double piston system. In an otherwise largely similar construction to the aforementioned embodiment, the medical syringe 1′ has, in addition to the piston 18, a further, upstream piston 80 inside the hollow body 6. A first active substance chamber 84 is formed through the intermediate space between the piston 18 and the further piston 80, and the second active substance chamber 86 is then located between the further piston 80 and the apical end 8 of the hollow body 6.

Such a double-chamber system is particularly used for liquid and/or lyophilized (freeze-dried) or powdered medicines that need to be dissolved before administration. Such double-chamber systems are thus a particularly suitable solution for lyophilized/liquid or liquid/liquid drug combinations. The system offers a variety of advantages for sensitive injectable medicines.

The solvent, for example, is kept in the first active substance chamber 84, whereas the actual, e.g. freeze-dried or powdered active substance is located in the second active substance chamber 86. When the active ingredient is administered, the solvent provided in the first active ingredient chamber 84 is first introduced into the second active ingredient chamber 86 by actuating the actuating plunger 4 via a bypass channel 90 arranged in the housing wall of the hollow body 6 and formed by a moulding 88 in the housing jacket, past the further plunger 80. There it dissolves the active substance held there so that it is ready for administration. Subsequently, by further actuation of the actuating plunger 4, the plunger 18 is moved up to the stop against the plunger 80, and then the plungers 18, 80 are moved further together, so that the nummore dissolved active substance located in the second active substance chamber 86 is dispensed via the needle 12.

As soon as the further plunger 80 reaches the needle 12 and the latter penetrates the plunger body, the remaining active substance is dispensed and then, after the position shown in FIG. 15, the retraction system is triggered according to the mode of operation described above. The reduction of the dead volume in the end region of the hollow body 6 is thereby made possible by a further formation 94 forming a bypass channel 92, through which the residual amount of active substance can flow towards the end 44 of the needle 12, comparable to the design already described above.

In this alternative embodiment of the medical syringe 1′, the needle holder 10 could also be made in one piece with the hollow body 6 forming the syringe housing. In the present embodiment example, which is considered to be independently inventive, however, the needle holder 10 is designed as a separate component and can be screwed to the hollow body 6 by means of a thread, in particular a Luer thread 100. Alternatively, the needle holder 10 could also be plugged or attachable to the hollow body 6 or the syringe cone forming the syringe housing.

### LIST OF REFERENCE SIGNS

1, 1′ Medical syringe
2 Cartridge or cartridge unit
4 Drive or actuation unit
6 Hollow body
8 distal end
10 Needle holder
12 Hollow needle
14 Bearing sleeve
15 Protective cap
16 proximal end
18 Piston
19 Receiving hole
20 Pusher plate
22 Operating plunger
24 End
26 Dome element
28 Shaft
30 Dome plate
32 Locking element
34 Spring
36 Protective housing
38 Safety spring
40 Needle tube
42,44 Needlepoint
46 Plastic sheath
48,50 Holding groove
52 Needle holder
54 Body
56 Holding bracket
58 Needle bearing
60 Piston skirt
62, 64 Shank rib
66 Return
68 Thigh
70 Support bead
72 Notch
80 Piston
84, 86 Active substance chamber
88,94 Shaping
90,92 Bypass channel
100 Luer thread
D maximum distance
d minimum distance
dE end distance

1. Syringe for administering a medicament dose to a patient, with a hollow body forming a syringe housing and containing a medicament active substance, at a distal end of which a hollow needle provided for injection of the medicament active substance is mounted in a bearing sleeve, and in which a displaceable piston provided at a proximal end with a receiving hole for the hollow needle is arranged, wherein the displaceable piston is constructed in several parts and, in addition to a needle holder having a holding bracket which carries a needle bearing forming the receiving hole for the hollow needle, comprises a piston jacket which is shaped in such a way that, when the needle holder is inserted, it leaves free on both sides of the holding bracket inflow channels into the enclosed end of the hollow needle for the medicament active substance.

2. The syringe of claim 1, wherein the holding bracket of the needle holder carries the needle bearing forming the receiving hole and is provided on its inside with a circumferential locking lip which, when the hollow needle is inserted into the needle bearing, engages in a retaining groove arranged on an outer circumference thereof and fixes the hollow needle in a longitudinal direction.

3. The syringe of claim 2, in which dimensions of the retaining groove and of the circumferential locking lip associated therewith in the needle bearing are selected in such a way, that, taking into account deformability of material at an outer circumference of the hollow needle and of material of the needle bearing surrounding it, a holding or breakaway force of the hollow needle thus engaged in the needle bearing is greater in the longitudinal direction than a corresponding holding or breakaway force of a holding groove of the hollow needle in the bearing sleeve.

4. The syringe of claim 1 with passive needle protection, which is provided with a spring triggering retraction of the hollow needle, wherein the spring, upon retraction of the hollow needle into an interior of the hollow body, additionally displaces a trigger plate provided for actuating the syringe in a direction away from the distal end of the syringe.

5. The syringe of claim 2 with passive needle protection, which is provided with a spring triggering retraction of the hollow needle, wherein the spring, upon retraction of the hollow needle into an interior of the hollow body, additionally displaces a trigger plate provided for actuating the syringe in a direction away from the distal end of the syringe.

6. The syringe of claim 3 with passive needle protection, which is provided with a spring triggering retraction of the hollow needle, wherein the spring, upon retraction of the hollow needle into an interior of the hollow body, additionally displaces a trigger plate provided for actuating the syringe in a direction away from the distal end of the syringe.
